(19) United States
(12) Patent Application Publication
(10) Pub. No.: US 2013/0108599 A1
(43) Pub. Date: May 2, 2013

(54) Herbal Vaginal Compositions

(71) Applicant: Tamyra Comeaux, Houston, TX (US)

(72) Inventor: Tamyra Comeaux, Houston, TX (US)

(21) Appl. No.: 13/286,096

(22) Filed: Oct. 31, 2011

(51) Int. Cl. A61K 36/82; A61P 31/00

(52) U.S. Cl. 424/93.45

(57) ABSTRACT

An herbal vaginal composition for treating vaginal infections while maintaining the natural vaginal flora, herbal ingredients comprising: coconut oil as the base ingredient, shea butter, neem oil, lavender oil, vitamin E, lactobacillus, green tea, tea tree, ylang ylang, sweet orange, myrrh, frankincense, nirvanas base, and clove oil.

### CROSS REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO A SEQUENCE LISTING

Not Applicable

### BACKGROUND OF THE INVENTION

1. Field of the Invention

This invention relates generally to treating vaginal infections, and in particular, to treating vaginal infections without disturbing the natural vaginal flora.

2. Description of Related Art

It is well known that in a female between the age of menarche and menopause, the normal vagina provides an ecosystem for a variety of microorganisms. Bacteria are the predominant type of microorganism present in the vagina; most women harbor about 10.sup.9 bacteria per gram of vaginal fluid. The bacterial flora of the vagina is comprised of both aerobic and anaerobic bacteria. The more commonly isolated bacteria are Lactobacillus species, Corynebacteria, Gardnerella vaginalis, Staphylococcus species, Peptococcus species, aerobic and anaerobic Streptococcus species, and Bacteroides species. Other microorganisms that have been isolated from the vagina on occasion include yeast (Candida albicans), protozoa (Trichomonas vaginalis), mycoplasma (Mycoplasma hominis), chlamydia (Chlamydia trachomatis), and viruses (e.g., Herpes simplex). These latter organisms are generally associated with vaginitis or venereal disease, although they may be present in low numbers without causing symptoms. Physiological, social, and idiosyncratic factors affect the quantity and species of bacteria present in the vagina. Physiological factors include age, day of the menstrual cycle, and pregnancy. For example, vaginal flora present in the vagina throughout the menstrual cycle can include lactobacilli, corynebacterium, ureaplasma, and mycoplasma. Social and idiosyncratic factors include method of birth control, sexual practices, systemic disease (e.g., diabetes), and medications.

Bacterial proteins and metabolic products produced in the vagina can affect other microorganisms and the human host. For example, the vagina between menstrual periods is mildly acidic having a pH ranging from about 3.8 to about 4.5. This pH range is generally considered the most favorable condition for the maintenance of normal vaginal flora. At that pH, the vagina normally harbors numerous species of microorganisms in a balanced ecology, playing a beneficial role in providing protection and resistance to infection and makes the vagina inhospitable to some species of bacteria such as Staphylococcus aureus (S. aureus). The low pH is a consequence of the growth of lactobacilli and their production of acidic products. Microorganisms in the vagina can also produce antimicrobial compounds such as hydrogen peroxide and bactericides directed at other bacterial species. One example is the lactocins, bacteriocin like products of lactobacilli directed against other species of lactobacilli. Some microbial products produced in the vagina may negatively affect the human host. For example, S. aureus is a bacteria that commonly colonizes human skin and mucous membranes. It causes disease in humans through invasion or through the production of toxic proteins.

During menses, the pH of the vagina moves toward neutral and can become slightly alkaline. This change permits microorganisms whose growth is inhibited by an acidic environment the opportunity to proliferate. For example, S. aureus is more frequently isolated from vaginal swabs during menstruation than from swabs collected between menstrual periods. When S. aureus is present in an area of the human body that harbors a normal microbial population such as the vagina, it may be difficult to eradicate the S. aureus bacteria without harming members of the normal microbial flora required for a healthy vagina Typically, antibiotics that kill S. aureus are not an option for use in catamenial products because of their effect on the normal vaginal microbial flora and their propensity to stimulate toxin production if all of the S. aureus are not killed. An alternative to eradication is technology designed to prevent or substantially reduce the bacteria's ability to produce toxins.

Vaginal infection is a clinical syndrome and exists in three primary forms, i.e., bacterial vaginosis, Candida vaginitis (“yeast”), and trichomonas vaginitis (“trich”). Bacterial vaginosis, for example, is a polymicrobial vaginal infection believed to be caused by an increase in the number of anaerobic organisms with a concomitant decrease in lactobacilli in the vagina. The decrease in the number of lactobacilli in the vagina has the dual effect of decreasing competition for nutrients and decreasing the amount of lactic acid present (i.e., increasing the pH). This allows for the multiplication of opportunistic pathogens in the vagina, whose growth is normally suppressed by the lactobacilli and the acidic pH of the vagina. The principal pathogen associated with bacterial vaginosis is believed to be Gardnerella vaginalis. Symptoms of bacterial vaginosis generally include an unpleasant smell, an elevated vaginal pH greater than about 5.0, a thin homogeneous discharge, and the presence of Gardnerella clue cells (i.e., vaginal epithelial cells coated with small Gram-variable rods).

Current treatment regimens for bacterial infection of the vagina involve the use of various broad spectrum antibiotics, such as metronidazole. However, antibiotics are often undesirable because they may kill a broad range of the normal bacterial flora in the vagina, including the beneficial lactobacilli. This may cause secondary complications, because the lactobacilli keep various opportunistic pathogens in the vagina from proliferating. The treatment may then necessitate a further treatment regimen, such as the ingestion of cultured dairy products to replace the lactobacilli in the body, as well as treatment by antifungal agents. Moreover, a rise in the level of anaerobes due to a lack of lactobacilli could further complicate the infection. Additionally, antibiotics, when used frequently within the vagina, may cause systemic toxicity through absorption from the vagina. In addition, trichomonas vaginitis (or “trich”) is one of the most common vaginal infections and is considered a sexually transmitted disease. Symptoms of trichomonas vaginitis include vulvar itching and odorous vaginal discharge. Trichomonas vaginitis is caused by Trichomonas vaginalis, a single-celled protozoan parasite not normally found in the flora of the genitourinary tract. Trichomonas vaginalis is a flagellate protozoa that is pear-shaped and about the size of a white blood cell.

These motile cells have four flagellae and a single nucleus. Like bacterial vaginosis, this pathology is generally treated with metronidazole.

Further, the yeast Candida albicans causes the disease known as candidiasis (or “thrush”), as well as vulvitis (or “vulval” infection). Candida albicans are present in most humans as a harmless commensal organism. Problems arise, however, when a person experiences a loss of normal bacterial flora. In severely immune compromised patients, for example, Candida albicans infection may spread throughout the body and cause systemic infections. Candidiasis is usually treated with fluconazole, but this may have serious side effects and is not recommended for use during pregnancy.

The prior art comprises active agents with pharmaceutical qualities that have been developed and approved for use in the treatment of afflictions of the vaginal cavity and the prevention of conception. These include fungicides, spermicides, etc. Although pharmaceutically active agents have been developed, it has been difficult to achieve optimal potential effectiveness from these agents. It has been found that gels, foams, creams, suppositories and tablets that are presently used as vaginal delivery systems break down almost immediately following insertion into the vaginal cavity and have minimal bioadherence to the vaginal walls. This is believed to be due to their water miscibility and/or their lack of physical stability at 37 degrees C. (body temperature). Thus, they exhibit limited effectiveness.

In this regard, some of the more conventional treatment regimens for various vaginal diseases have been described as follows: Candida is treated topically with miconazole 2% or clotrimazole 1% cream, vaginal tablets, or suppositories for three to seven days. Trichomonas is treated with metronidazole 250 mg rid or 500 mg bid orally for five days; 2 gm in a single daily dose may be used. Ideally, the sexual partner should also be treated. Gardnerella or anaerobic infections are treated similarly to Trichomonas, with metronidazole. About 25% of patients have recurrences and require re-treatment in two to three months. Lowering vaginal pH with propionic acid jelly has also been suggested. Chlamydial infections are treated with doxycycline 100 mg bid or erythromycin 500 mg qid orally for seven days. Mycoplasma is treated with doxycycline 100 mg bid orally for ten days. For any of these infections, sexual partners should be treated simultaneously, if possible. Conventional treatments thus have two important negative characteristics. Some of the treatments described are systemic, such as oral metronidazole for trichomonas or bacterial vaginosis and tetracycline for chlamydia. Systemic treatment may have systemic side effects. The second difficulty is that none of these treatments or procedures attempt to normalize the vaginal flora growth habitat by either maintaining or enhancing acceptable microbial growth patterns.

In light of the foregoing, it would be further appreciated to develop a therapeutically effective treatment for vaginal infections that maintain the natural flora of the vagina.

### BRIEF SUMMARY OF THE INVENTION

Specifically, the present invention is directed at the objectives of relieving the symptoms of vaginal infections including inflammation, irritation, dryness, discharge, and unpleasant odor;

Another objective is to therapeutically treat common vaginal infections including Bacterial Vaginosis, Candida Vaginitis, Trichomonas Vaginitis, and Chlamydia;
Another objective is to therapeutically treat uncommon vaginal infections including vaginal eczema;
Another objective is to develop effective suppositories in treating infections that do not break down almost immediately following insertion into the vaginal cavity, and have suitable bioadherence to the vaginal walls;
Another objective is to develop such suppositories that also do not destroy the protective mucus lining of the vaginal walls;

Another objective is to develop compositions that can be used externally on the skin and in the anus of both males and females.

Another objective is to develop suppositories meeting the above objectives while avoiding the side effects resulting from using antibiotics to treat vaginal infections; i.e., between menses maintaining a pH between 3.8 to about 4.5; and maintaining or enhancing microbial growth patterns in the vagina.

The present invention is directed toward compositions and methods of making and using such compositions to achieve the objectives above. The present invention comprises herbal compositions prepared with a base derived from coconut and palm oils in such proportions so that the resulting compositions do not burn or otherwise irritate the vagina. An exemplary composition includes coconut oil, shea butter, neem oil, lavender oil, vitamin E, lactobacillus, green tea, tea tree, ylang ylang, sweet orange, myrrh oil, and frankincense. An exemplary composition includes 16 ounces of coconut oil, 2 tablespoons of shea butter, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang ylang, 1 teaspoon of sweet orange, ½ teaspoon myrrh oil, and ½ teaspoon of frankincense. 1 scoop of lactobacillus is equal to 1 teaspoon containing 1.5 billion units of lactobacillus acidphilus.

Another example composition also directed at the above objectives comprises 16 ounces of coconut oil, ½ teaspoon of neem oil, a teaspoon of tea tree, a tablespoon of pomegranate shea butter, 1 tablespoon of vitamin E, 2 teaspoons of lavender oil, ½ tablespoon of green tea, and a ½ tablespoon of lactobacillus. One method of making the compositions is to combine and mix the ingredients until the green tea and lactobacillus powders thoroughly blend with the other ingredients. At room temperature the composition can be conveniently applied externally to the skin. Another method of making the compositions is to first combine the ingredients, heat the combined ingredients, then mix the ingredients until the green tea and lactobacillus powders thoroughly blend with the other ingredients. To prepare suppositories for vaginal or anal use, refrigerate the blended ingredients, which will harden evenly. Keep suppositories refrigerated until use.

Other objects and advantages of the present invention will be readily apparent upon a reading of the following detailed descriptions of preferred embodiments of the invention and the appended claims.

### DETAILED DESCRIPTION OF THE INVENTION

In the following description, the present invention is directed toward compositions and methods of making and using compositions for treating vaginal infections while maintaining the natural vaginal flora. A preferred embodiment of the present invention comprises compositions prepared with herbal ingredients. The ingredients include 16 ounces of coconut oil, 2 tablespoons of shea butter, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang ylang, 1 teaspoon of sweet orange, ½ teaspoon myrrh oil, a ½ teaspoon of frankincense, and nirvanas base (½ teaspoon is preferred). To make the composition in the preferred embodiment, first combine the ingredients, heat the combined ingredients, then mix the ingredients until the green tea and lactobacillus powders thoroughly blend with the other ingredients. To prepare suppositories for vaginal or anal use, refrigerate the blended ingredients, which will harden evenly. Keep suppositories refrigerated until use. For best results, use almond shea butter, and heat the ingredients in a conventional microwave for 50 seconds.

Another preferred embodiment of the present invention comprises an herbal composition including 16 ounces of coconut oil, rosemary jasmine (½ teaspoon is preferred), tea green (½ teaspoon is preferred), a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang ylang, 1 teaspoon of sweet orange, ½ teaspoon myrrh oil, a ½ teaspoon of frankincense, and nirvanas base (½ teaspoon is preferred). Still another preferred embodiment of the suppository includes 16 ounces of coconut oil, ½ teaspoon of neem oil, a teaspoon of tea tree, a tablespoon of pomegranate shea butter, 1 tablespoon of vitamin E, 2 teaspoons of lavender, ½ tablespoon of green tea, and a ½ tablespoon of lactobacillus. Adding clove oil (½ teaspoon of is preferred) to any of the above preferred embodiments improves the therapeutic effectiveness of the compositions.

Each herbal composition ingredient has properties that treat the symptoms of vaginal infections pointed out earlier. Some properties are not commonly known. The compositions are a natural deodorizer, because the ingredients are natural and the combined amounts and proportions of the ingredients smell pleasant, which is unknown in the prior art. Cold-pressed Coconut oil contains capric/caprylic acid and lauric acid (fatty acids) both of which have antiviral, antifungal, antimicrobial, and anti-inflammatory properties. Organic Coconut oil is rich in medium-chain fatty acids (MFCA) that destroy free radicals—-harmful compounds that trigger skin inflammation that also promote the development of skin cancer. The moment organic Coconut oil is applied to the skin, the microbes on the skin will react with the MFCA, transforming the microbes into free fatty acids that help reduce the chronic inflammation that patients with eczema and psoriasis suffer.

Coconut oil is a lubricant, promotes good hydration, and soothes the vagina reducing irritation common to vaginal infections. Coconut oil also ensures the other ingredients are more evenly dispersed in the body. Coconut oil is also highly resistant to rancidity which preserves the integrity of the herbal ingredients and prolongs the shelf life of the compositions of the present invention. The anti-bacterial and anti-fungal power of coconut oil can treat diaper rash and other skin irritations. Through substantial testing and experimentation, the inventor has discovered that when the other ingredients are blended with coconut oil in amounts and proportions exemplified above, the resulting compositions do not burn or irritate the vagina. And remarkably, the compositions synergistically comprise the therapeutic effectiveness of the individual herbal ingredients and sustains the natural vaginal flora.

It is known that Lavender oil is a good deodorizer, but in the prior art it is not known that Lavender oil combined with the other exemplified herbal ingredients above would eliminate odor associated with common vaginal infections. Lavender Oil can help to relieve the pain and itch and provide some stress relief which boosts the immune system helping to heal the lesions associated with Psoriasis and Eczema. Lavender Oil due to its antiseptic, antibacterial, anti-fungal and circulatory stimulating properties has been used as a treatment for Eczema. It is also an immune booster and powerful anti-inflammatory for the skin. All these qualities help to speed healing and prevent or reduce scarring of the skin. Lavender works to relieve the associated pain and itch that frequently occurs with skin inflammation. However, it is unknown in the prior art the Lavender oil can be used to treat vaginal infections generally, and vaginal eczema in particular. However, through substantial testing and experimentation, the inventor has discovered using Lavender oil in the amounts and proportions exemplified above enhances the therapeutic effectiveness of the compositions of the present invention.

Tea tree oil contains antibacterial properties that help reduce naturally occurring bacteria on the skin. Tea tree oil also helps soothe irritated skin caused by excessive scratching or chaffing. The medicinal benefits of tea tree oil are effective against psoriasis, Candida, skin infections, hemorrhoids, nail infections, acne and dandruff. Tea tree and green tea help manage bacteria and viruses. The antifungal activity in tea tree and coconut oil relieve yeast infection, which is a fungus. At room temperature the compositions of the present invention, as exemplified above, will melt. Placing the melted compositions on affected skin relieves irritation. Placing the melted compositions on boils regularly will eliminate boils, by killing the bacteria in boils.

Lactobacillus is found in a healthy vagina. As stated, 1 scoop of lactobacillus is equal to 1 teaspoon containing 1.5 billion units of lactobacillus acidphilus. The addition of lactobacillus in the compositions of the present invention, in the amounts and proportions exemplified above, helps to normalize the vaginal flora, because the lactobacilli keep various opportunistic pathogens in the vagina from proliferating by lowering the vagina pH. Lactobacillus acidphilus is acidic. The vaginal mucus instantly absorbs the lactobacillus, which immediately adapts to the floral environment of the vagina, resulting in the proliferation of the lactobacillus, which result lowers the vagina pH.

Clove oil has extraordinary properties and many uses; however, it is not commonly known in the prior art that Clove oil can be used to treat vaginal infections. Used in isolation or as a base Clove oil would burn the vagina. Clove oil is a powerful antioxidant, antiseptic, and is a pain reliever. Clove oil stimulates circulation, and blood flow to the skin. Clove oil has the following prior art uses:

Athletics foot, nail fungus, & skin problems: Clove oil is just as effective as oil of oregano in treating these conditions. It can be applied directly to the skin or nails, but if skin is sensitive or broken it must be diluted with extra virgin olive oil or coconut oil.
Antioxidant: Clove essential oil is the powerful antioxidant of any fruit or herb.
Antiseptic Uses Clove oil is the active ingredient in several mouthwashes and a number of over-the-counter toothache pain-relief preparations.
Cold Extremities Clove oil stimulates circulation, and blood flow to the skin, making it very useful for people who have cold extremities.
Colds, Flu, Bronchitis, Fever & Whooping Cough: Cloves fights germs, viruses and bacteria, and it encourages the loosening of phlegm from the respiratory system. It also promotes sweating with fevers, colds, and flu, which is very healing. It is often used in remedies for whooping cough.
Depression: This powerful herb also has the ability to relieve depression.
Digestive Aid: Like many culinary spices, clove may help relax the smooth muscle lining of the digestive tract. A few drops of the oil in water will stop vomiting, and clove tea will relieve diarrhea, gas, bloating, intestinal spasms and nausea.
Eyes: Clove also helps prevent the breakdown in the retina of the eye, which slows down macular degeneration and aids vision in old age.
Fatigue & Drowsiness: Researchers found that sniffing the spicy aroma of cloves reduces drowsiness, irritability, and headaches. It stimulates the mind, increases memory recall, and relieves mental fatigue.
Headache: One drop of clove oil applied to the roof of the mouth can instantly relieve many headaches.
Insect Repellent Clove, when used with citrus oils, is an effective insect repellent!
Pain: Long used in aromatherapy to relieve pain, it is also uplifting oil with a delightful scent. For general pain relief, add 3 drops of clove oil to 1 teaspoon of coconut oil or extra virgin olive oil. It is also used topically to relieve general aches and pains.
Toothache, oral hygiene: Dentists use clove oil as an oral anesthetic. They also use it to disinfect root canals. Clove oil will stop the pain of a toothache when dropped into a cavity. For temporary relief of toothache, dip a cotton swab in clove oil and apply it to the affected tooth and surrounding gum.
According to a study at the University of Iowa, compounds in clove oil have shown “strong activity” against bacteria associated with plaque formation and gum disease and it treats mouth sores and ulcers, and sore gums.
Warts: A few drops of clove oil soaked into a band aid and applied to warts has been known to dissolve warts.
Cautions: Clove oil is not recommended for pregnant or nursing mothers. Medicinal amounts of clove should not be given to children under age 2. Clove oil may be diluted in extra virgin olive oil and coconut oil. Those who are taking blood-thinner medications should not take clove oil because it, too, is a powerful blood-thinner. Concentrated doses of Clove oil may cause stomach upset.
Antioxidant Activity of Clove Essential Oil: Many foods and substances contain antioxidants which absorb free radicals (toxins). Scientists at Tufts University have developed a scale for the U.S. Department of Agriculture called the ORAC (Oxygen Radical Absorption Capacity) test. The higher the ORAC score is, the more capable that particular food, spice or herb is of destroying free radicals (antioxidant).
Essential oils have the highest ORAC scores of any known substance. According to the Essential Oils Desk Reference, 2nd ED., they are as follows:

### Essential Oil (Botanical Name) ORAC Score

| | |
|---|---|
| Roman Chamomile (Chamaemelum nobile) | 2,446 |
| Juniper (Juniperus osteosperma) | 2,517 |
| Spearmint (Mentha spicata) | 5,398 |
| Lemongrass (Cymbopogen flexuosus) | 17,765 |
| Eucalyptus (Eucalyptus (Eucalyptus globulus) | 24,157 |
| Cinnamon Bark (Cinnamamum verum) | 103,448 |
| Mountain Savory (Satureja montana) | 113,071 |
| Oregano (Origanum compactum) | 153,007 |
| Thyme (Thymus vulgaris) | 159,590 |
| Clove (Syzygium aromaticum) | 10,786,875 |

These essential oils are even more potent than fruits which are promoted as powerful antioxidants, such as blueberries, ORAC Score 2,400. The last six oils listed above are even more powerful than Chinese wolfberries, ORAC score 23,300. Clove has an ORAC score over 10 million: That means a drop of Clove Oil contains 400 times more antioxidants per unit volume than wolfberries, the most powerful of all known fruits, and a 15 ml bottle of Clove Oil has the antioxidant capacity of 40 quarts of blueberries.

In one study, the researchers evaluated the antimicrobial activity of clove oil against a range of fungal pathogens including that responsible for urogenital infection. Clove oil was found to possess strong antifungal activity against opportunistic fungal pathogens such as Candida albicans, Cryptococcus neoformans and Aspergillus fumigatus, etc. The Clove oil was found to be extremely successful in the treatment of experimental murine vaginitis in model animals. On evaluating various formulations, topical administration of the liposomized clove oil was found to be most effective against treatment of vaginal candidiasis.

Frankincense resin is distilled by steam or CO2 to extract its precious essential oil, which is used extensively in modern aromatherapy. This oil is rejuvenating to the skin, treating acne, bacterial and fungal infections, and to treat wounds and scars. Thus, it is used in cosmetics, soaps, and perfumes. However, Frankincense is not known to be used as a treatment of vaginal infection. The therapeutic properties of Myrrh oil are: antiseptic, anti-microbial, anti-phlogistic, anti-inflammatory, astringent, balsamic, carminative, cicatrizing, diuretic, expectorant, fungicidal, stimulant, stomach and tonic. Myrrh oil is effective against excessive mucus in the lungs; it helps to clear ailments such as colds, catarrh, coughs, sore throats and bronchitis. Myrrh oil is helpful for diarrhea, dyspepsia, flatulence and hemorrhoids. Myrrh oil is very good for mouth and gum disorders such as mouth ulcers, pyorrhea, gingivitis, spongy gums and sore throats. For the skin Myrrh oil could be useful for the treatment of boils, skin ulcers, bedsores, chapped and cracked skin, and ringworm, weeping wounds eczema and athlete's foot. Myrrh oil could be of great help in cases of scanty periods, leucorrhoea, thrush and amenorrhea.

The therapeutic properties of Ylang ylang oil are: aphrodisiac, antidepressant, antiseptic, hypotensive and sedative. Ylang ylang oil has a euphoric and sedative effect on the nervous system; it can help with anxiety, tension, shock, fear and panic. Its aphrodisiac qualities may be of use in impotence and frigidity. Ylang ylang oil can be particularly useful with rapid breathing and rapid a heartbeat; it can also help with reducing high blood pressure. It can be useful for intestinal infections. Ylang ylang oil could have a soothing effect on the skin and its stimulating effect on the scalp could promote more luxurious hair growth. However, it is not known in the prior art that Ylang ylang oil is capable of being used in the delicate flora of the vagina.

The use of shea butter in the compositions of the present invention is twofold. The shea butter is used to give the suppositories firmness. She Butter also has therapeutic benefits. Shea Butter can provide relief from many ailments, from dry skin to many minor dermatological diseases. Shea Butter has been clinically shown to provide the following benefits for the skin: Daily skin moisturizer (face and body); Dry skin relief; Dry scalp; Skin rash-including diaper rash; Skin peeling, after tanning; Blemishes and wrinkles; Itching skin due to dryness; Sunburn; Shaving cream to reduce razor irritation; Small skin wounds; Skin cracks; Soften tough skin on feet (especially heels); Stretch mark prevention during pregnancy; Minor burns; Eczema; Sun and wind protection; Even skin tone; Reduces blemishes and scarring; Eliminating scalp irritation from dryness or chemical processing; Preventing bumps after shaving; Reducing acne (especially in combination with African Black Soap); Absorbs quickly without leaving a greasy residue; Helps restore elasticity to skin; and Restores luster to hair. The properties of shea butter contribute to the therapeutic effectiveness of the compositions of the present invention in treating vaginal eczema.

Shea Butter nourishes the skin with Vitamins A, E and F. Vitamins A and E help maintain the skin and keep it clear and healthy. These vitamins are particularly helpful for sun damaged skin. These vitamins help prevent premature wrinkles and facial lines. Vitamin F acts as a skin protector and rejuvenator. Vitamin F soothes rough, dry or chapped skin and helps soften dry or damaged hair. Shea Butter is high in unsaponifiables (a type of fat). Shea Butter has between 7-12% unsaponifiables. For comparison, avocado oil, a well known skin conditioner, has between 2-6%. This high level of unsaponifiables is one of the properties that enables Shea Butter to treat the conditions of the skin (e.g., Psoriasis, Eczema) and vaginal eczema. Also, Shea Butter easily penetrates the skin allowing the skin to breathe, and not clogging pores. Shea Butter has a high level of cinnamic acid, a natural sun screen. So, it provides some degree of protection from the sun. Shea Butter is also anti-inflammatory making it useful in treating rheumatism. This property of Shea Butter contributes to the compositions of the present invention in reducing inflammation associated with some vaginal infections, as described above.

Through testing and experimentation, the inventor has discovered that the compositions of the present invention are effective in treating minor skin conditions, including vaginal eczema. Several of the inventor's patients who have used Shea Butter for eczema and/or psoriasis have asserted Shea Butter works as well as steroids at a fraction of the cost and without the side effects. One patient is allergic to the sun. When such patient began using Shea Butter lotion (not even pure Shea Butter), she accidentally discovered that it prevented the rash she normally would get from even a small amount of exposure to the sun. Shea Butter provides moisture to dry or damaged hair from the roots to the very tips, repairing and protecting against weather damage, dryness and brittleness. Shea Butter also absorbs quickly and completely into the scalp to rehydrate without clogging pores. Shea Butter is particularly beneficial for processed and heat-treated hair. Shea Butter is an excellent treatment for dry scalp. Shea Butter restores luster to damaged hair.

All Shea Butter is not the same. Shea Butter loses some of its healing properties as it sits on the shelf, so very old Shea Butter is not as beneficial. Refining techniques will vary. Highly processed Shea Butter will not be as effective. Sometimes Shea Butter is mixed with other ingredients that reduce its benefits. Shea Butter is not very expensive. Obtain Shea Butter only from reputable sources to ensure freshness, quality and the refining technique to ensure there are no contaminants and the active ingredients in Shea Butter are left in.

Green tea extract (GTE), including catechins and caffeine, have strong anti-microbial activity. One study determined the anti-microbial and anti-fungal effects of a GTE on the vaginal pathogens, Proteusmirabilis (KCTC 2510), Streptococcus pyogenes (KCTC 3096), and Candida albicans (KCTC 7270). The study concluded the growth inhibitory effects of a GTE and the ethyl acetate fraction against P. mirabilis and S. pyogenes were stronger compared to the anti-microbial activity of the H2O fraction. Among the catechins, epigallocatechin gallate had the strongest anti-microbial activity. The growth of C. albicans was strongly inhibited by the methylene chloride fraction and caffeine; the anti-fungal activity of a GTE was due to caffeine.

The study also evaluated whether the in vitro activity of a GTE included purified solvent fractions stable to heat and pH changes. The stability of transtype catechins increased following heat treatment, but this did not significantly affect the anti-microbial activity. A GTE were stable over the pH range 4-10. The most active anti-microbial activity of a GTE occurred in an alkaline, rather than an acid environment, except for the anti-microbial activity against S. pyogenes and C. albicans, which was not influenced by pH. Taken together, the results of this study indicated that a GTE may be a valuable therapeutic agent for the treatment of vaginal pathogens.

Neem oil has been traditionally used against viral infections and the research results so far are very promising. There has been a scientific study in 1997 where researchers tested the effect of neem against the herpes simplex virus-2, and found Neem “provided significant protection”. That study was done in mice and the study tested neem's efficiency in preventing vaginal transmission of genital herpes. The way neem was used in that study is similar to how one would use a vaginal contraceptive. However, preventing transmission is not the same as treating an existing infection. There have been other studies with other viruses; also, some where researchers treated existing infections (but not herpes). Heretofore scientists have not been able to exactly ascertain how Neem works, but Neem seems to make it difficult for viruses to reproduce. This means the impact of any viral infection will be a lot less. Neem also boosts the immune system and that also helps to fight off the virus.

Most studies involving neem and viruses have been done in vitro, i.e., in a test tube. Studies on live animals used mostly neem leaf extract at the highest non-toxic concentration. Definitively, these studies are the only documented clinical studies. There is no research data that proves neem cures herpes. There are some reports that neem does indeed speed up the healing of cold sores (herpes simplex type 1, not genital herpes). Herpes is usually treated both internally with neem leaf (either as tea or in capsule form, neem leaf tea is usually more effective) and externally by treating the lesions with neem leaf paste, a neem based cream or neem seed oil at least once a day. Evidence suggests that neem boosts the immune system (like neem does when used internally), which is desirable in managing genital herpes and avoiding outbreaks. Neem should not be taken internally by pregnant women or women trying to conceive. However, topical application of neem is not harmful.

Nirvanas base is Polyethylene Glycol Polymers, which have received much attention as suppository bases in recent years because they possess many desirable properties. They are chemically stable, nonirritating, miscible with water and mucous secretions, and can be formulated, either by molding or compression, in a wide range of hardness and melting point. Like glycerinated gelatin, Nirvanas does not melt at body temperature, but dissolve to provide a more prolonged release than theobroma oil. Consequently, Nirvanas base, blended with the other ingredients of the compositions exemplified above, reduces the melting rate of the compositions, yet is readily absorbed by the secretions and lining in the vagina. Through substantial experimentation and testing, the inventor has discovered the appropriate amounts and proportions of the ingredients exemplified above, so that the rate of melting of the compositions approximates the rate of absorption of the exemplified compositions by the vagina. Because of this discovery the compositions of the present invention leak very little when used as suppositories, as exemplified above.

Rosemary Oil is a colorless or pale yellow mobile fluid with a strong, fresh, minty-herbaceous scent and a woody-balsamic undertone. It blends well with frankincense, lavender, citronella, oregano, thyme, pine, basil, peppermint, labdanum, elemi, cedarwood, petitgrain, cinnamon and other spice oils. The general actions of Rosemary Oil are: Analgesic, anti-oxidant, antirheumatic, antispasmodic, aphrodisiac, astringent, carminative, cephalic, cholagogue, choleretic, cicatrizant, cordial, cytophylactic, diaphoretic, digestive, diuretic, emmenagogue, fungicidal, hepatic, hypertensive, parasiticide, restorative, rubefacient, stimulant (circulatory, adrenal cortex, hepatobiliary), stomachic, sudorific, tonic (nervous, general), vulnerary. Jasmine oil is extracted from Jasminum Gradiflora (synonym officinale), of the Oleaceae family and is also known as jasmin, jessamine and common jasmine. There are well over 100 constituents found in jasmine oil, but the main chemical components are benzyl acetate, linalool, benzyl alcohol, indole, benzyl benzoate, cis-jasmone, geraniol, methyl anthranilate and trace amounts of p. cresol, farnesol, cis-3-hexenyl benzoate, eugenol, nerol, ceosol, benzoic acid, benzaldehyde, y-terpineol, nerolidol, isohytol, phytol etc. The therapeutic properties of jasmine oil are anti-depressant, antiseptic, aphrodisiac, anti-spasmodic, cicatrisant, expectorant, galactagogue, parturient, sedative and uterine. Sweet orange has the following characteristics: antidepressant, anti-inflammatory, antiseptic, bactericidal, fungicidal, hypotensive, nervous sedative, lymphatic stimulant, tonic-digestive

The compositions are easy to use to treat vaginal infections. By preparing the compositions in the preferred embodiments above, the suppositories slowly melt at body temperature when inserted into the vagina. The suppositories prepared in the above preferred embodiments will absorb well in the vagina, decreasing leakage. Keep the suppositories refrigerated until use. The blended compositions or melted down suppositories, as described above, can be applied directly to the genitals of males, who are sexual partners of women with vaginal infections. Such applications to male sexual partners will prevent re-infection. After applying to males, as stated, rinsing after 20 minutes is optional. For female use, for best results apply at night when supine, so that in case the suppositories melt faster than they are absorbed by the vagina, the melting suppository will flow toward the cervix. However, as stated above, the compositions will leak very little when used as suppositories, because in the amounts and proportions of the blended ingredients, as exemplified above, the rate of absorption of the compositions by the vagina approximates the rate of melting of the compositions while in the vagina.

The compositions cannot be used by pregnant women or women who are breastfeeding. The compositions can only be used by females 13 or older. For best results, use one suppository (refrigerated compositions as described above) nightly for 3-14 days for acute infections. For chronic or chronic recurring infections, insert one suppository nightly for 2-4 weeks. The suppositories are prophylactic in preventing vaginal infections caused by physiological factors, such as age, day of the menstrual cycle; and in preventing vaginal infections caused by idiosyncratic factors such as method of birth control, systemic disease (e.g., diabetes), and medications. To prevent vaginal infections, use one suppository daily, at bedtime, during menses, for four consecutive months. For anal irritation, these suppositories may be used rectally.

These compositions (blended ingredients) and suppositories, when melted down as described above, have a number of beneficial uses unknown to the average member in the field of invention. The blended ingredients or melted down suppositories can be used to eliminate dandruff, by lubricating the scalp. Recurrent application of the blended ingredients or melted down suppositories will cause the hair on the scalp to thicken. Application to the skin will cure Eczema due to dryness of the skin. Applying the melted down suppositories to razor bumps will eliminate razor bumps.

All of the above descriptions are merely illustrative of the many applications of the composition of matter of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

What is claimed is:

1. A composition of matter, the active ingredients including herbal ingredients, herbal ingredients comprising: coconut oil, shea butter, neem oil, lavender oil, vitamin E, lactobacillus, green tea, tea tree, ylang ylang oil, sweet orange, myrrh oil, and frankincense; said herbal ingredients, wherein coconut oil is the base ingredient, are combined and blended in such amounts and proportions, so that the therapeutic effectiveness of the respective herbal ingredients synergistically combine to effectively treat common vaginal infections.

2. The composition in claim 1, wherein the blended herbal ingredients are included in such amounts and proportions, so that after being refrigerated until hard, the melting rate of the composition when inserted into the vagina of a user approximates the rate of absorption of the composition by the vagina.

3. A composition of matter the active ingredients including herbal ingredients, herbal ingredients comprising: 16 ounces of coconut oil, 2 tablespoons of shea butter, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang, 1 teaspoon of sweet orange, ½ teaspoon myrrh, and ½ teaspoon of frankincense.

4. The composition of matter in claim 3, wherein the type of shea butter is almond shea butter.

5. The composition of matter in claim 4, including ½ teaspoon of nirvanas base.

6. The composition of matter in claim 5, wherein the composition is prepared by first combining the ingredients, heating the combined ingredients, then mixing the ingredients until the green tea and lactobacillus powders thoroughly blend with the other ingredients.

7. The composition of matter in claim 6, wherein the composition is prepared for vaginal or anal use, by refrigerating the blended ingredients, which will harden evenly.

8. The composition of matter claim in claim 7, wherein the heating step comprises, microwaving the ingredients for 50 seconds.

9. A vaginal suppository prepared with herbal ingredients, ingredients comprising: 16 ounces of coconut oil, 2 tablespoons of almond shea butter, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang ylang, 1 teaspoon of sweet orange, ½ teaspoon myrrh, a ½ teaspoon of frankincense, and ½ teaspoon of nirvanas base; the ingredients are prepared by combing the ingredients, microwaving the ingredients for 50 seconds, then mixing the ingredients.

10. A vaginal suppository prepared with herbal active ingredients, active ingredients comprising: 16 ounces of coconut oil, ½ teaspoon of rosemary jasmine, ½ teaspoon of tea green, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang, 1 teaspoon of sweet orange, ½ teaspoon myrrh, a ½ teaspoon of frankincense, and nirvana's base; the ingredients are prepared by combing the ingredients, microwaving the ingredients for 50 seconds, then mixing the ingredients.

11. A vaginal suppository prepared with herbal ingredients, ingredients comprising: 16 ounces of coconut oil, ½ teaspoon of neem oil, a teaspoon of tea tree, a tablespoon of pomegranate shea butter, 1 tablespoon of vitamin E, 2 teaspoons of lavender, ½ tablespoon of green tea, and a ½ tablespoon of lactobacillus.

12. The suppository in claim 11, wherein the suppository is prepared by combining the ingredients, heating the ingredients, then mixing the ingredients.

13. The suppository in claim in claim 12, wherein the heating step comprises, microwaving the ingredients for 50 seconds.

14. A coconut oil base vaginal suppository for treating vaginal infections prepared with herbal ingredients, the suppository is prepared by:
the step of combining 16 ounces of coconut oil, 2 tablespoons of almond shea butter, a ½ teaspoon of neem oil, 2 teaspoons of lavender oil, 1 tablespoon of vitamin E, 1 scoop of lactobacillus, 1 teaspoon of green tea, 1 teaspoon of tea tree, ½ teaspoon of ylang, 1 teaspoon of sweet orange, ½ teaspoon myrrh, a ½ teaspoon of frankincense, and ½ teaspoon of nirvanas base;
the step of microwaving the ingredients for 50 seconds, and
the step of mixing the ingredients.

15. A coconut oil base vaginal suppository for treating vaginal infections prepared with herbal ingredients, the suppository is prepared by:
the step of combining 16 ounces of coconut oil, ½ teaspoon of neem oil, a teaspoon of tea tree, a tablespoon of pomegranate shea butter, 1 tablespoon of vitamin E, 2 teaspoons of lavender, ½ tablespoon of green tea, and a ½ tablespoon of lactobacillus;
the step of microwaving the ingredients for 50 seconds, and
the step of mixing the ingredients.

16. The method in claim 14, wherein the step of combining ingredients includes, combing ½ teaspoon of clove oil.

17. The method in claim 15, wherein the step of combining ingredients includes, combing ½ teaspoon of clove oil.
